(19) United States
(12) Patent Application Publication
(10) Pub. No.: US 2026/0194430 A1
(43) Pub. Date: Jul. 9, 2026

(54) WELD COUPON DESIGN

(71) Applicant: GM Global Technology Operations LLC, Detroit, MI (US)

(72) Inventors: Hui-ping Wang, Troy, MI (US); Guangze Li, Novi, MI (US); Eric Gregory Hartnagle, Rochester Hills, MI (US); Alexander Mepham, Clarkston, MI (US); Jacob Wayne Disbro, Troy, MI (US); Min Liu, Shanghai (CN)

(21) Appl. No.: 19/009,463

(22) Filed: Jan. 3, 2025

(51) Int. Cl. G01N 3/06; G01N 3/08; G01N 33/207

(52) U.S. Cl. CPC G01N 3/066; G01N 3/08; G01N 33/207; G01N 2203/0017; G01N 2203/0282; G01N 2203/0617

(57) ABSTRACT

A weld coupon for testing weld integrity includes a top blank and a bottom blank in overlapping engagement with one another and connected to one another with an electrical weld, each of the top blank and bottom blank adapted to uniformly pass electrical current applied to the weld coupon across the weld and to uniformly pass tensile load applied to the weld coupon across the weld.

### INTRODUCTION

The present invention relates generally to weld coupons used to test a specific weld. Weld coupons generally comprise two pieces of material that are welded together, and after being welded together, are tested to determine mechanical or electrical properties thereof. For example, to test tensile strength of a weld, two pieces are welded together to form a weld coupon whereupon, tensile force is applied to the weld coupon, applied in opposite directions to the two welded pieces, to evaluate the integrity of the weld under tensile loading. Further, to test electrical conductivity of a weld, and electrical current is passed through the weld coupon, whereupon the resistance of the weld is measured based on potential drop of the current between opposite ends of the weld coupon.

Weld coupons have specific design criteria depending upon the type of testing. Weld coupons designed to test tensile integrity of a weld have different design criteria than weld coupons designed to test electrical conductivity of the weld. Thus, while current weld coupon designs achieve their intended purpose, there is a need for a new and improved weld coupon that is designed for both testing of weld integrity under tensile loading and electrical conductivity of the weld.

### SUMMARY

According to several aspects of the present disclosure, a weld coupon for testing weld integrity includes a top blank and a bottom blank in overlapping engagement with one another and connected to one another with an electrical weld, each of the top blank and bottom blank adapted to uniformly pass electrical current applied to the weld coupon across the weld and to uniformly pass tensile load applied to the weld coupon across the weld.

According to another aspect, the weld coupon further includes an overlap zone defined by the overlapping engagement of the top blank and the bottom blank, the electrical weld positioned within the overlap zone, each of the top blank and the bottom blank including a loading zone positioned at an end distal from the weld and adapted to support application of an electrical current from the loading zone of the top blank, across the weld, to the loading zone of the bottom blank, and support application of a tensile load from the loading zone of the top blank, across the weld, to the loading zone of the bottom blank, and a distribution zone extending from the weld to the loading zone and adapted to uniformly pass applied electrical current between the loading zone and the weld, and uniformly pass applied tensile load between the loading zone and the weld.

According to another aspect, the loading zone of the top blank has a length that is equal to a length of the loading zone of the bottom blank.

According to another aspect, the distribution zone of the top blank has a length that is equal to a length of the distribution zone of the bottom blank.

According to another aspect, the loading zone of each of the top blank and the bottom blank has a length that is greater than or equal to both a minimum length adapted to allow tensile loading to be applied to the weld coupon, and a minimum length adapted to allow electrical current to be applied to the weld coupon.

According to another aspect, for each of the top blank and the bottom blank, a distance from the loading zone to a boundary of the overlap zone is adapted to uniformly distribute applied tensile load between the loading zone and the weld across a width of the weld coupon.

According to another aspect, each of the top blank and the bottom blank includes an equipotential zone extending from the loading zone, wherein, within the equipotential zone electrical current applied between the loading zone and the weld is uniformly distributed across the width of the weld coupon.

According to another aspect, the distribution zone of each of the top blank and the bottom blank has a length that is greater than or equal to both a sum of the distance from the loading zone to the boundary of the overlap zone and a distance from the boundary of the overlap zone to the weld, and a sum of a length of the equipotential zone and a distance from the equipotential zone to the weld.

According to another aspect, for each of the top blank and the bottom blank, the distance from the loading zone to the boundary of the overlap zone is at least ten millimeters.

According to another aspect, for each of the top blank and the bottom blank, the length of the equipotential zone is greater than the distance from the loading zone to the boundary of the overlap zone.

According to another aspect, for each of the top blank and the bottom blank, the equipotential zone defines a region for application of external probes for measuring potential drop of an electrical current passing through the weld.

According to another aspect, for each of the top blank and the bottom blank, the distance from the loading zone to the boundary of the overlap zone is greater than or equal to the width of the weld coupon and less than or equal to twice the width of the weld coupon.

According to another aspect, a length of the overlap zone is the sum of twice the distance between the boundary of the overlap zone to the weld and a length of the weld.

According to another aspect, an overall length of each of the top blank and the bottom blank is equal to a sum of the length of the overlap zone, the length of the loading zone and the distance from the loading zone to the boundary of the overlap zone.

According to another aspect, an overall length of the weld coupon is equal to a sum of the length of the overlap zone, the length of the loading zone of the top blank, the length of the loading zone of the bottom blank, the distance from the loading zone of the top blank to the boundary of the overlap zone and the distance from the loading zone of the bottom blank to the boundary of the overlap zone.

According to several aspects of the present disclosure, a method of using a weld coupon for testing weld integrity, wherein the weld coupon includes a top blank and a bottom blank in overlapping engagement with one another and connected to one another with an electrical weld, each of the top blank and bottom blank adapted to uniformly pass an electrical current applied to the weld coupon across the weld and to uniformly pass a tensile load applied to the weld coupon across the weld, the method including passing an electrical current through the weld coupon and measuring a potential drop of the electrical current across the weld, and applying a tensile load to the weld coupon.

According to another aspect, the weld coupon further includes an overlap zone defined by the overlapping engagement of the top blank and the bottom blank, the electrical weld positioned within the overlap zone, each of the top blank and the bottom blank including a loading zone positioned at an end distal from the weld and having a length that is greater than or equal to both a minimum length adapted to allow tensile loading to be applied to the weld coupon, and a minimum length adapted to allow electrical current to be applied to the weld coupon, and adapted to support application of an electrical current from the loading zone of the top blank, across the weld, to the loading zone of the bottom blank, and support application of a tensile load from the loading zone of the top blank, across the weld, to the loading zone of the bottom blank, and a distribution zone extending from the weld to the loading zone and adapted to uniformly pass applied electrical current between the loading zone and the weld, and uniformly pass applied tensile load between the loading zone and the weld, wherein a distance from the loading zone to a boundary of the overlap zone adapted to uniformly distribute applied tensile load between the loading zone and the weld across a width of the weld coupon, and each of the top blank and the bottom blank includes an equipotential zone extending from the loading zone, wherein, within the equipotential zone electrical current applied between the loading zone and the weld is uniformly distributed across the width of the weld coupon, the passing an electrical current through the weld coupon and measuring a potential drop of the electrical current across the weld further including applying a first electrical probe to the top blank at a position adjacent to the boundary of the overlap zone on a first side of the weld, applying a second electrical probe to the bottom blank at a position adjacent to the boundary of the overlap zone on a second side of the weld, passing an electrical current between the loading zone of the top blank and the loading zone of the bottom blank, across the weld, and measuring a potential drop between the first electrical probe and the second electrical probe.

According to another aspect, the distribution zone of each of the top blank and the bottom blank has a length that is greater than or equal to both a sum of the distance from the loading zone to the boundary of the overlap zone and a distance from the boundary of the overlap zone to the weld, and a sum of a length of the equipotential zone and a distance from the equipotential zone to the weld, and, wherein, the passing an electrical current through the weld coupon and measuring a potential drop of the electrical current across the weld further includes uniformly passing the electrical current applied to the weld coupon across the distribution zone between the loading zone and the weld, and the applying a tensile load to the weld coupon further includes uniformly passing tensile load applied to the weld coupon across the distribution zone between the loading zone and the weld.

Further areas of applicability will become apparent from the description provided herein. It should be understood that the description and specific examples are intended for purposes of illustration only and are not intended to limit the scope of the present disclosure.

### BRIEF DESCRIPTION OF THE DRAWINGS

The drawings described herein are for illustration purposes only and are not intended to limit the scope of the present disclosure in any way.

FIG. 1 is a schematic perspective view of a weld coupon according to an exemplary embodiment of the present disclosure;

FIG. 2 is a top view of the weld coupon shown in FIG. 1;

FIG. 3 is a side view of a weld coupon illustrating design characteristics specific for electrical testing of the weld coupon;

FIG. 4 is a side view of a weld coupon illustrating design characteristics specific for testing the weld coupon under tensile loading;

FIG. 5 is a schematic flow chart illustrating a method according to an exemplary embodiment of the present disclosure; and

FIG. 6 is a schematic flow chart illustrating a method according to another exemplary embodiment of the present disclosure.

The figures are not necessarily to scale and some features may be exaggerated or minimized, such as to show details of particular components. In some instances, well-known components, systems, materials or methods have not been described in detail in order to avoid obscuring the present disclosure. Therefore, specific structural and functional details disclosed herein are not to be interpreted as limiting, but merely as a basis for the claims and as a representative basis for teaching one skilled in the art to variously employ the present disclosure.

### DETAILED DESCRIPTION

The following description is merely exemplary in nature and is not intended to limit the present disclosure, application, or uses. Furthermore, there is no intention to be bound by any expressed or implied theory presented in the preceding technical field, background, brief summary or the following detailed description. It should be understood that throughout the drawings, corresponding reference numerals indicate like or corresponding parts and features. Although the figures shown herein depict an example with certain arrangements of elements, additional intervening elements, devices, features, or components may be present in actual embodiments. It should also be understood that the figures are merely illustrative and may not be drawn to scale.

Example embodiments are provided so that this disclosure will be thorough, and will fully convey the scope to those who are skilled in the art. Numerous specific details are set forth such as examples of specific compositions, components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, elements, compositions, steps, integers, operations, and/or components, but do not preclude the presence or addition of one or more other features, integers, steps, operations, elements, components, and/or groups thereof. Although the open-ended term “comprising,” is to be understood as a non-restrictive term used to describe and claim various embodiments set forth herein, in certain aspects, the term may alternatively be understood to instead be a more limiting and restrictive term, such as “consisting of” or “consisting essentially of” Thus, for any given embodiment reciting compositions, materials, components, elements, features, integers, operations, and/or process steps, the present disclosure also specifically includes embodiments consisting of, or consisting essentially of, such recited compositions, materials, components, elements, features, integers, operations, and/or process steps. In the case of “consisting of,” the alternative embodiment excludes any additional compositions, materials, components, elements, features, integers, operations, and/or process steps, while in the case of “consisting essentially of” any additional compositions, materials, components, elements, features, integers, operations, and/or process steps that materially affect the basic and novel characteristics are excluded from such an embodiment, but any compositions, materials, components, elements, features, integers, operations, and/or process steps that do not materially affect the basic and novel characteristics can be included in the embodiment.

Any method steps, processes, and operations described herein are not to be construed as necessarily requiring their performance in the particular order discussed or illustrated, unless specifically identified as an order of performance. It is also to be understood that additional or alternative steps may be employed, unless otherwise indicated.

When a component, element, or layer is referred to as being “on,” “engaged to,” “connected to,” or “coupled to” another element or layer, it may be directly on, engaged, connected or coupled to the other component, element, or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various steps, elements, components, regions, layers and/or sections, these steps, elements, components, regions, layers and/or sections should not be limited by these terms, unless otherwise indicated. These terms may be only used to distinguish one step, element, component, region, layer or section from another step, element, component, region, layer or section. Terms such as “first,” “second,” and other numerical terms when used herein do not imply a sequence or order unless clearly indicated by the context. Thus, a first step, element, component, region, layer or section discussed below could be termed a second step, element, component, region, layer or section without departing from the teachings of the example embodiments.

Spatially or temporally relative terms, such as “before,” “after,” “inner,” “outer,” “beneath,” “below,” “lower,” “above,” “upper,” and the like, may be used herein for ease of description to describe one element or feature's relationship to another element(s) or feature(s) as illustrated in the figures. Spatially or temporally relative terms may be intended to encompass different orientations of the device or system in use or operation in addition to the orientation depicted in the figures.

Throughout this disclosure, the numerical values represent approximate measures or limits to ranges to encompass minor deviations from the given values and embodiments having about the value mentioned as well as those having exactly the value mentioned. Other than in the working examples provided at the end of the detailed description, all numerical values of parameters (e.g., of quantities or conditions) in this specification, including the appended claims, are to be understood as being modified in all instances by the term “about” whether or not “about” actually appears before the numerical value. “About” indicates that the stated numerical value allows some slight imprecision (with some approach to exactness in the value; approximately or reasonably close to the value; nearly). If the imprecision provided by “about” is not otherwise understood in the art with this ordinary meaning, then “about” as used herein indicates at least variations that may arise from ordinary methods of measuring and using such parameters. For example, “about”, with reference to percentages, comprises a variation of plus/minus 5%, “about”, with reference to temperatures, comprises a variation of plus/minus five degrees, and “about”, with reference to distances, comprises plus/minus 10%. In addition, disclosure of ranges includes disclosure of all values and further divided ranges within the entire range, including endpoints and sub-ranges given for the ranges. In addition, disclosure of ranges includes disclosure of all values and further divided ranges within the entire range, including endpoints and sub-ranges given for the ranges.

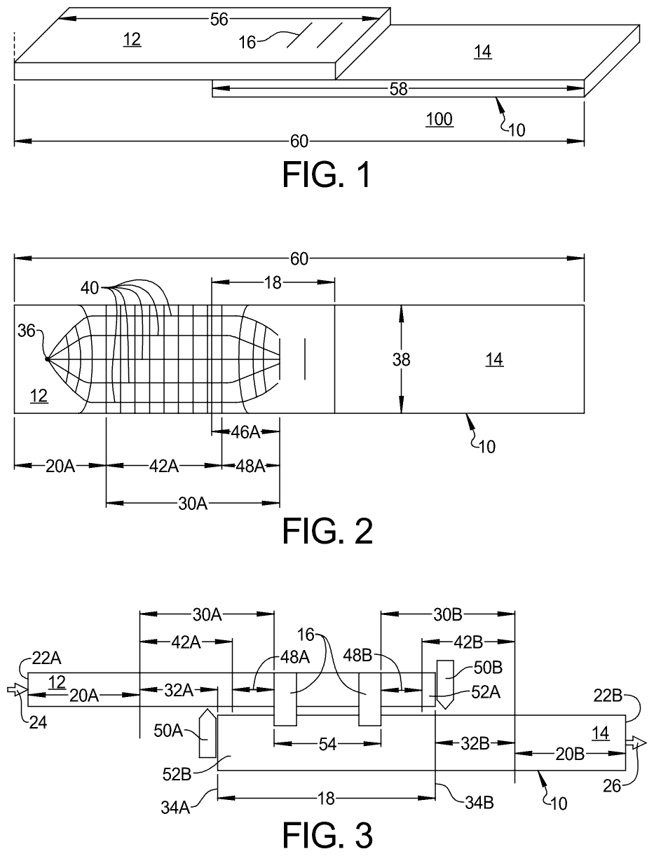

Example embodiments will now be described more fully with reference to the accompanying drawings. Referring to FIG. 1 and FIG. 2, a weld coupon 10 for testing weld integrity includes a top blank 12 and a bottom blank 14 in overlapping engagement with one another and connected to one another with an electrical weld 16. Each of the top blank 12 and bottom blank 14 are adapted to uniformly pass electrical current applied to the weld coupon 10 across the weld 16 and to uniformly pass tensile load applied to the weld coupon 10 across the weld 16. Thus, the weld coupon 10 is equally suited for testing the integrity of the weld 16 under tensile loading conditions and for testing electrical conductivity of the weld 16. Here the weld 16 being illustrated has a geometry of double straight lines but can have any geometry such as a single straight line, a circular line, a spot weld.

The weld coupon 10 is designed to allow testing of a specific weld 16. Thus, the material used for the top blank 12 and the bottom blank 14 are based on the specific application for which the weld 16 will be used. In this way, a specific type of weld between two specific materials or two pieces of the same material, can be evaluated for suitability for a particular application.

The weld coupon 10 includes an overlap zone 18 defined by the overlapping engagement of the top blank 12 and the bottom blank 14. The electrical weld 16 is positioned within the overlap zone 18, thus interconnecting the top blank 12 and the bottom blank 14. Each of the top blank 12 and the bottom blank 14 includes a loading zone 20A, 20B positioned at an end 22A, 22B distal from the weld 16. As shown, the top blank 12 has a loading zone 20A located at an end 22A of the top blank 12 distal from the weld 16, and the bottom blank 14 has a loading zone 20B located at an end 22B of the bottom blank 14 distal from the weld 16. The loading zones 20A, 20B are adapted to support application of an electrical current from the loading zone 20A of the top blank 12, across the weld 16, to the loading zone 20B of the bottom blank 14, and support application of a tensile load from the loading zone 20A of the top blank 12, across the weld 16, to the loading zone 20B of the bottom blank 14.

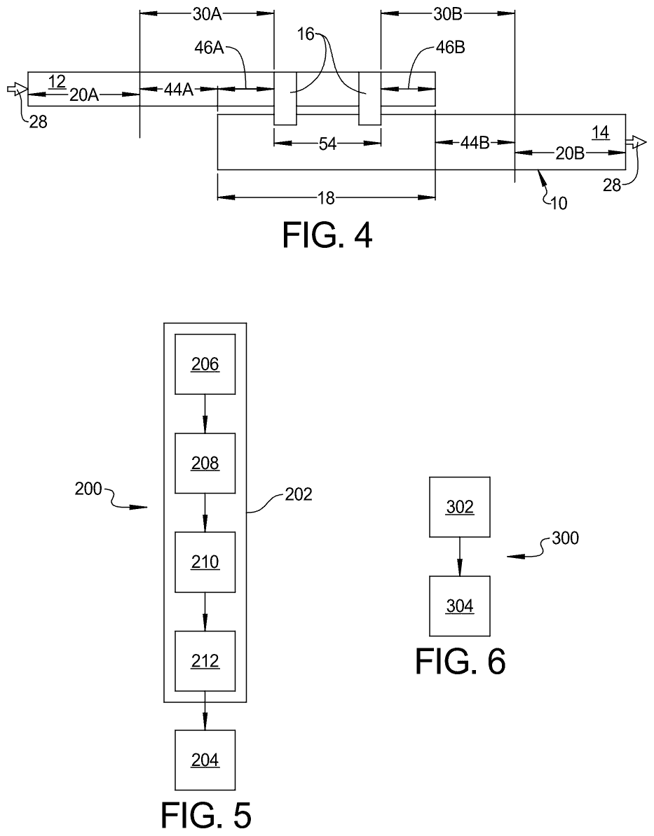

The loading zones 20A, 20B are adapted to allow the weld coupon 10 to be inserted and secured within a testing unit. In an exemplary embodiment, the loading zone 20A of the top blank 12 and the loading zone 20B of the bottom blank 14 are equal in length. Referring to FIG. 3, the loading zones 20A, 20B are adapted to allow an electrical current to pass into the weld coupon 10, through the loading zone 20A of the top blank 12, as indicated by arrow 24, pass through the weld coupon 10, and the weld 16 between the top blank 12 and the bottom blank 14, and to exit the weld coupon 10, through the loading zone 20B of the bottom blank 14, as indicated by arrow 26. The current flow can be a direct flow from top to bottom or vice versa. It can also be an alternate flow. Referring to FIG. 4, the loading zones 20A, 20B are adapted to allow tensile forces to be applied to the top blank 12 and the bottom blank 14, as indicated by arrows 28.

To allow the weld coupon 10 of the present disclosure to be suitable for either testing under tensile loading or testing for electrical conductivity, the loading zone 20A, 20B of each of the top blank 12 and the bottom blank 14 has a length that is greater than or equal to both a minimum length adapted to allow tensile loading to be applied to the weld coupon 10, and a minimum length adapted to allow electrical current to be applied to the weld coupon 10. A testing unit for testing the weld coupon 10 under tensile loading will require a minimum length of the loading zones 20A, 20B to allow the testing unit to clamp onto the top blank 12 and the bottom blank 14 and apply tensile forces, as indicated by arrows 28. A testing unit for testing the electrical conductivity of the weld 16 of the weld coupon 10 will require a minimum length of the loading zones 20A, 20B to allow the testing unit to connect with and pass electrical current into and through the weld coupon 10, as indicated by arrows 24, 26. Thus, to ensure that the weld coupon 10 is suitable for both, the loading zones 20A, 20B of the top blank 12 and the bottom blank 14 have a length that is at least equal to the larger of a minimum length required for tensile testing and a minimum length required for application of an electrical current.

Each of the top blank 12 and the bottom blank 14 further includes a distribution zone 30A, 30B extending from the weld 16 to the loading zone 20A, 20B. The top blank 12 includes a distribution zone 30A that extends from the loading zone 20A of the top blank 12 to the weld 16 and the bottom blank 14 includes a distribution zone 30B that extends from the loading zone 20B of the bottom blank 14 to the weld 16. In an exemplary embodiment, the distribution zone 30A of the top blank 12 and the distribution zone 30B of the bottom blank 14 are equal in length. The distribution zones 30A, 30B are adapted to uniformly pass applied electrical current between the loading zones 20A, 20B and the weld 16, and to uniformly pass applied tensile loading between the loading zones 20A, 20B and the weld 16.

Referring to FIG. 2 and FIG. 3, the distribution zone 30A of the top blank 12, when testing the electrical conductivity of the weld coupon 10, is a distance that provides a length 32A of the top blank 12 between the loading zone 20A and a boundary 34A of the overlap zone 18 to allow electrical current applied at a point contact 36 within the loading zone 20A of the top blank 12 to spread uniformly across a width 38 of the top blank 12, pass through the top blank 12 and reconcentrate at the weld 16, as indicated by current lines 40 in FIG. 2. Thus, within the distribution zone 30A of the top blank 12, an equipotential zone 42A is defined which extends from the loading zone 20A of the top blank 12, and within which, current applied to the top blank 12 within the loading zone 20A is evenly distributed across the width 38 of the top blank 12 and the weld coupon 10.

Further, the distribution zone 30B of the bottom blank 14, when testing the electrical conductivity of the weld coupon 10, is a distance that provides a length 32B of the bottom blank 14 between the loading zone 20B of the bottom blank 14 and a boundary 34B of the overlap zone 18 to allow electrical current passing through the weld 16 to spread uniformly across a width 38 of the bottom blank 14, pass through the bottom blank 14 and reconcentrate at a point contact (not shown) within the loading zone 20B of the bottom blank 14, and out of the weld coupon 10. Thus, within the distribution zone 30B of the bottom blank 14, an equipotential zone 42B is defined which extends from the loading zone 20B of the bottom blank 14, and within which, current passing through the bottom blank 14 is evenly distributed across the width 38 of the bottom blank 14 and the weld coupon 10.

The length of the distribution zones 30A, 30B necessary for the electrical current to spread uniformly across the width 38 of the top blank 12 and the bottom blank 14 is dependent upon the material or materials of the top blank 12 and the bottom blank 14, dimensional features such as thickness of the top blank 12 and the bottom blank 14, and the type and magnitude of the electrical current being passed therethrough. Thus, the length of the distribution zones 30A, 30B of the top blank 12 and the bottom blank 14, are calculated based on at least the factors mentioned above.

Referring to FIG. 4, the distribution zone 30A of the top blank 12, when testing the weld coupon 10 under tensile loading, is a distance that provides a length 44A of the top blank 12 between the loading zone 20A and the boundary 34A of the overlap zone 18 to allow tensile stress applied at the loading zones 20A, 20B of the top blank 12 and the bottom blank 14 to distribute uniformly across the width 38 of the top blank 12, pass across the top blank 12 and act uniformly on the weld 16. Further, the distribution zone 30B of the bottom blank 14, when testing the weld coupon 10 under tensile loading, is a distance that provides a length 44B of the bottom blank 14 between the loading zone 20B and the boundary 34B of the overlap zone 18 to allow tensile stress applied at the loading zones 20A, 20B of the top blank 12 and bottom blank 14 to distribute uniformly across the width 38 of the bottom blank 14, pass across the bottom blank 14 and act uniformly on the weld 16. Thus, ensuring that the tensile loading is applied evenly across the weld 16.

The length of the distribution zones 30A, 30B necessary for the tensile stress to be spread uniformly across the width 38 of the top blank 12 and the bottom blank 14 is dependent upon the material or materials of the top blank 12 and the bottom blank 14, dimensional features such as thickness of the top blank 12 and the bottom blank 14, and the magnitude of the tensile force being applied. Thus, the length of the distribution zones 30A, 30B of the top blank 12 and the bottom blank 14 are calculated based on at least the factors mentioned above.

To allow the weld coupon 10 of the present disclosure to be suitable for either testing under tensile loading or testing for electrical conductivity, the distribution zone 30A of the top blank 12 has a length that is greater than or equal to both a sum of the distance 44A from the loading zone 20A to the boundary 34A of the overlap zone 18 and a distance 46A from the boundary 34A of the overlap zone 18 to the weld 16, and a sum of a length of the equipotential zone 42A and a distance 48A from the equipotential zone 42A to the weld 16, and the distribution zone 30B of the bottom blank 14 has a length that is greater than or equal to both a sum of the distance 44B from the loading zone 20B to the boundary 34B of the overlap zone 18 and a distance 46B from the boundary 34B of the overlap zone 18 to the weld 16, and a sum of a length of the equipotential zone 42B and a distance 48B from the equipotential zone 42B to the weld 16.

A minimum length is required to allow tensile loads to be uniformly distributed across the width 38 of the weld coupon 10 and thus, evenly applied to the weld 16. A minimum length is required to allow applied electrical current to spread uniformly across the width 38 of the weld coupon 10. Thus, to ensure the weld coupon 10 is suitable for both, the distribution zones 30A, 30B of the top blank 12 and the bottom blank 14 must be at least as long as the larger of the two.

The purpose of the equipotential zones 42A, 42B is to allow electrical probes 50A, 50B to be placed in contact with the top blank 12 and the bottom blank 14, wherein a potential drop of the electrical current passing through the weld coupon 10 can be measured between the two electrical probes 50A, 50B. The probes 50A, 50B must be applied at a point where the electrical current is uniform. For each of the top blank 12 and the bottom blank 14, the equipotential zone 42A, 42B defines a region for application of external electrical probes 50A, 50B for measuring the potential drop of the electrical current passing through the weld 16. In an exemplary embodiment, for each of the top blank 12 and the bottom blank 14, the distance 32A, 32B from the loading zone to the boundary 34A, 34B of the overlap zone 18 is at least ten millimeters, to provide sufficient space for application of the electrical probes 50A, 50B.

In an exemplary embodiment, for each of the top blank 12 and the bottom blank 14, the length of the equipotential zone 42A, 42B is greater than the distance 32A, 32B from the loading zone 20A, 20B to the boundary 34A, 34B of the overlap zone 18. Thus ensuring that the equipotential zone 42A, 42B extends from the loading zones 20A, 20B, beyond the boundaries 34A, 34B of the overlap zone 18. For consistency, repeated testing must be conducted with the electrical probes 50A, 50B placed in the same position within the equipotential zones 42A, 42B of weld coupons. Thus, the outer boundaries 34A, 34B of the overlap zone 18 provide a easily repeatable stepped profile, wherein an external electrical probe 50A is placed in contact with the top blank 12 adjacent a distal end 52B of the bottom blank 14 at the boundary 34B of the overlap zone 18, and another external electrical probe 50B is placed in contact with the bottom blank 14 adjacent a distal end 52A of the top blank 12 at the boundary 34A of the overlap zone 18, as shown in FIG. 3.

In another exemplary embodiment, for each of the top blank 12 and the bottom blank 14, the distance 32A, 32B from the loading zone 20A, 20B to the boundary 34A, 34B of the overlap zone 18 is greater than or equal to the width 38 of the weld coupon 10 and less than or equal to twice the width 38 of the weld coupon 10.

In another exemplary embodiment, a length of the overlap zone 18 of the weld coupon 10 is the sum of twice the distance between the boundary 34A, 34B of the overlap zone 18 to the weld 16 and a length of the weld 54, an overall length 56, 58 of each of the top blank 12 and the bottom blank 14 is equal to a sum of the length of the overlap zone 18, the length of the loading zone 20A, 20B and the distance 32A, 32B from the loading zone 20A, 20B to the boundary 34A, 34B of the overlap zone 18, and an overall length 60 of the weld coupon 10 is equal to a sum of the length of the overlap zone 18, the length of the loading zone 20A of the top blank 12, the length of the loading zone 20B of the bottom blank 20B, the distance 32A from the loading zone 20A of the top blank 12 to the boundary 34A of the overlap zone 18 and the distance 32B from the loading zone 20B of the bottom blank 14 to the boundary 34B of the overlap zone 18.

As shown in FIG. 3 and FIG. 4, there is a gap present between the top blank 12 and the bottom blank 14, wherein the top blank 12 and the bottom blank 14 are interconnected only at the weld 16. It should be understood by those skilled in the art that the gap is not necessary, and not intentionally designed into the weld coupon 10 of the present disclosure. The process of forming the weld 16 between the top blank 12 and the bottom blank 14 may, depending on the specifics of the welding process, cause such a gap to form between the top blank 12 and the bottom blank 14.

Further, as shown in the Figures, the weld 16 comprises two parallel weld lines. It should be understood by those skilled in the art that the novel features of the present disclosure are applicable to weld coupons 10 having a single weld point, a single weld line, multiple weld points or multiple weld lines that are either perpendicular or parallel to the orientation of the weld coupon 10, or any other weld characteristics that may be utilized.

Referring to FIG. 5, a method 200 of using a weld coupon 10 for testing weld integrity, wherein the weld coupon 10 includes a top blank 12 and a bottom blank 14 in overlapping engagement with one another and connected to one another with an electrical weld 16, each of the top blank 12 and bottom blank 14 adapted to uniformly pass an electrical current applied to the weld coupon 10 across the weld 16 and to uniformly pass tensile load applied to the weld coupon 10 across the weld 16, the method 200 comprising, starting at block 202, passing an electrical current through the weld coupon 10 and measuring a potential drop of the electrical current across the weld 16, and, moving to block 204, applying a tensile load to the weld coupon 10.

In an exemplary embodiment, the weld coupon 10 further includes an overlap zone 18 defined by the overlapping engagement of the top blank 12 and the bottom blank 14, the electrical weld 16 positioned within the overlap zone 18. Each of the top blank 12 and the bottom blank 14 include a loading zone 20A, 20B positioned at an end 22A, 22B distal from the weld 16 and having a length that is greater than or equal to both a minimum length adapted to allow tensile loading to be applied to the weld coupon 10, and a minimum length adapted to allow electrical current to be applied to the weld coupon 10, and adapted to support application of an electrical current from the loading zone 20A of the top blank 12, across the weld 16, to the loading zone 20B of the bottom blank 14, and support application of a tensile load from the loading zone 20A of the top blank 12, across the weld 16, to the loading zone 20B of the bottom blank 14.

Each of the top blank 12 and the bottom blank 14 include a distribution zone 30A, 30B extending from the weld to the loading zone and adapted to uniformly pass applied electrical current between the loading zones 20A, 20B and the weld 16, and uniformly pass applied tensile load between the loading zones 20A, 20B and the weld 16, wherein a distance 44A, 44B from the loading zone 20A, 20B to a boundary 34A, 34B of the overlap zone 18 is adapted to uniformly distribute applied tensile load between the loading zones 20A, 20B and the weld 16 across a width 38 of the weld coupon 10, and each of the top blank 12 and the bottom blank 14 includes an equipotential zone 42A, 42B extending from the loading zones 20A, 20B, wherein, within the equipotential zones 42A, 42B electrical current applied between the loading zones 20A, 20B and the weld 16 is uniformly distributed across the width 38 of the weld coupon 10, the passing an electrical current through the weld coupon 10 and measuring a potential drop of the electrical current across the weld 16 at block 202 further including, moving to block 206, applying a first electrical probe 50A to the top blank 12 at a position adjacent to the boundary 34A of the overlap zone 18 on a first side of the weld 16, moving to block 208, applying a second electrical probe 50B to the bottom blank 14 at a position adjacent to the boundary 34B of the overlap zone 18 on a second side of the weld 16, moving to block 210, passing an electrical current from the loading zone 20A of the top blank 12, across the weld 16 and to the loading zone 20B of the bottom blank 14, and, moving to block 212, measuring a potential drop between the first electrical probe 50A and the second electrical probe 50B.

In another exemplary embodiment, the distribution zone 30A, 30B of each of the top blank 12 and the bottom blank 14 has a length that is greater than or equal to both a sum of the distance 44A, 44B from the loading zone 20A, 20B to the boundary 34A, 34B of the overlap zone 18 and a distance 46A, 46B from the boundary 34A, 34B of the overlap zone 18 to the weld 16, and a sum of a length of the equipotential zone 42A, 42B and a distance 48A, 48B from the equipotential zone 42A, 42B to the weld 16, and wherein, the passing an electrical current through the weld coupon 10 and measuring a potential drop of the electrical current across the weld 16 at block 202 further includes uniformly passing the electrical current applied to the weld coupon 10 across the distribution zone 30A, 30B between the loading zone 20A, 20B and the weld 16, and, the applying a tensile load to the weld coupon 10 at block 204 further includes uniformly passing tensile load applied to the weld coupon 10 across the distribution zone 30A, 30B between the loading zone 20A, 20B and the weld 16.

Testing of the weld coupon 10 comprises determining a resistance of the weld 16 between the top blank 12 and the bottom blank 14 by analysis of the measured potential drop between electrical probes 50A, 50B in contact with the top blank 12 and the bottom blank 14. Testing of the integrity of the weld 16 of the weld coupon 10 under tensile loading may involve visual analysis of the weld 16 after application of tensile loading and/or application of increasing tensile loading up to deformation and/or failure of the weld 16.

Referring to FIG. 6, a method 300 of forming a weld coupon 10 for testing weld integrity includes, beginning at block 302, forming a top blank 12 and a bottom blank 14, wherein each of the top blank 12 and the bottom blank 14 includes a loading zone 20A, 20B and a distribution zone 30A, 30B, and, moving to block 304, attaching, with an electrical weld 16, the top blank 12 and the bottom blank 14 in overlapping engagement with one another, the overlapping engagement defining an overlap zone 18 of the weld coupon 10, wherein, for each of the top blank 12 and the bottom blank 14, the loading zone 20A, 20B is adapted to support application of an electrical current from the loading zone 20A of the top blank 12, across the weld 16, to the loading zone 20B of the bottom blank 14, and support application of a tensile load from the loading zone 20A of the top blank 12, across the weld 16, to the loading zone 20B of the bottom blank 14, and the distribution zones 30A, 30B extending from the weld 16 to the loading zone 20A, 20B and adapted to uniformly pass applied electrical current between the loading zones 20A, 20B and the weld 16, and uniformly pass applied tensile load between the loading zones 20A, 20B and the weld 16, the forming the top blank 12 and the bottom blank 14 at block 302 further including forming the top blank 12 and the bottom blank 14 wherein the loading zone 20A, 20B has a length that is greater than or equal to both a minimum length adapted to allow tensile loading to be applied to the weld coupon 10, and a minimum length adapted to allow electrical current to be applied to the weld coupon 10, the loading zone 20A of the top blank 12 has a length that is equal to a length of the loading zone 20B of the bottom blank 14, and the distribution zone 30A of the top blank 12 has a length that is equal to a length of the distribution zone 30B of the bottom blank 14, a distance 44A, 44B from the loading zone 20A, 20B to a boundary 34A, 34B of the overlap zone 18 is adapted to uniformly distribute applied tensile load between the loading zone 20A, 20B and the weld 16 across a width 38 of the weld coupon 10, each of the top blank 12 and the bottom blank 14 includes an equipotential zone 42A, 42B extending from the loading zone 20A, 20B, wherein, within the equipotential zone 42A, 42B electrical current applied between the loading zone 20A, 20B and the weld 16 is uniformly distributed across the width 38 of the weld coupon 10, and the distribution zone 30A, 30B of each of the top blank 12 and the bottom blank 14 has a length that is greater than or equal to both a sum of the distance 32A, 32B from the loading zone 20A, 20B to the boundary 34A, 34B of the overlap zone 18 and a distance 46A, 46B from the boundary of the overlap zone 18 to the weld 16, and a sum of a length of the equipotential zone 42A, 42B and a distance 48A, 48B from the equipotential zone 42A, 42B to the weld 16.

In another exemplary embodiment, the forming the top blank 12 and the bottom blank 14 at block 302 further includes forming the top blank 12 and the bottom blank 14, wherein, for each of the top blank 12 and the bottom blank 14, the distance 32A, 32B from the loading zone 20A, 20B to the boundary 34A, 34B of the overlap zone 18 is at least ten millimeters, for each of the top blank 12 and the bottom blank 14, the length of the equipotential zone 42A, 42B is greater than the distance 44A, 44B from the loading zone 20A, 20B to the boundary 34A, 34B of the overlap zone 18, for each of the top blank 12 and the bottom blank 14, the equipotential zone 42A, 42B defines a region for application of external probes 50A, 50B for measuring potential drop of an electrical current passing through the weld 16, for each of the top blank 12 and the bottom blank 14, the distance 32A, 32B, 44A, 44B from the loading zones 20A, 20B to the boundary 34A, 34B of the overlap zone 18 is greater than or equal to the width 38 of the weld coupon 10 and less than or equal to twice the width 38 of the weld coupon 10, a length of the overlap zone 18 is the sum of twice the distance between the boundary 34A, 34B of the overlap zone 18 to the weld 16 and a length 54 of the weld 16, an overall length 56, 58 of each of the top blank 12 and the bottom blank 14 is equal to a sum of the length of the overlap zone 18, the length of the loading zone 20A, 20B and the distance 32A, 32B from the loading zone 20A, 20B to the boundary 34A, 34B of the overlap zone 18, and an overall length 60 of the weld coupon 10 is equal to a sum of the length of the overlap zone 18, the length of the loading zone 20A of the top blank 12, the length of the loading zone 20B of the bottom blank 14, the distance 32A from the loading zone 20A of the top blank 12 to the boundary 34A of the overlap zone 18 and the distance 32B from the loading zone 20B of the bottom blank 14 to the boundary 34B of the overlap zone 18.

A weld coupon 10 of the present disclosure offers several advantages. These include the ability to test the integrity of a weld under tensile loading and test electrical conductivity of the weld using the same weld coupon 10. This allows testing and design of welds without the necessity of having different weld coupons for testing the integrity of a weld under tensile loading and for testing electrical conductivity. Further, the design of the weld coupon 10 of the present disclosure provides a stepped profile that allows easily repeatable placement of electrical probes 50A, 50B for testing electrical conductivity of the weld 16.

The description of the present disclosure is merely exemplary in nature and variations that do not depart from the gist of the present disclosure are intended to be within the scope of the present disclosure. Such variations are not to be regarded as a departure from the spirit and scope of the present disclosure.

What is claimed is:

1. A weld coupon for testing weld integrity, comprising:
a top blank and a bottom blank in overlapping engagement with one another and connected to one another with an electrical weld, each of the top blank and bottom blank adapted to uniformly pass electrical current applied to the weld coupon across the weld and to uniformly pass tensile load applied to the weld coupon across the weld.

2. The weld coupon of claim 1, further including:
an overlap zone defined by the overlapping engagement of the top blank and the bottom blank, the electrical weld positioned within the overlap zone;
each of the top blank and the bottom blank including:
a loading zone positioned at an end distal from the weld and adapted to:
support application of an electrical current from the loading zone of the top blank, across the weld, to the loading zone of the bottom blank; and
support application of a tensile load from the loading zone of the top blank, across the weld, to the loading zone of the bottom blank; and
a distribution zone extending from the weld to the loading zone and adapted to:
uniformly pass applied electrical current between the loading zone and the weld; and
uniformly pass applied tensile load between the loading zone and the weld.

3. The weld coupon of claim 2, wherein the loading zone of the top blank has a length that is equal to a length of the loading zone of the bottom blank.

4. The weld coupon of claim 3, wherein the distribution zone of the top blank has a length that is equal to a length of the distribution zone of the bottom blank.

5. The weld coupon of claim 4, wherein the loading zone of each of the top blank and the bottom blank has a length that is greater than or equal to both:
a minimum length adapted to allow tensile loading to be applied to the weld coupon; and
a minimum length adapted to allow electrical current to be applied to the weld coupon.

6. The weld coupon of claim 5, wherein for each of the top blank and the bottom blank, a distance from the loading zone to a boundary of the overlap zone is adapted to uniformly distribute applied tensile load between the loading zone and the weld across a width of the weld coupon.

7. The weld coupon of claim 6, wherein each of the top blank and the bottom blank includes an equipotential zone extending from the loading zone, wherein, within the equipotential zone electrical current applied between the loading zone and the weld is uniformly distributed across the width of the weld coupon.

8. The weld coupon of claim 7, wherein the distribution zone of each of the top blank and the bottom blank has a length that is greater than or equal to both:
a sum of the distance from the loading zone to the boundary of the overlap zone and a distance from the boundary of the overlap zone to the weld; and
a sum of a length of the equipotential zone and a distance from the equipotential zone to the weld.

9. The weld coupon of claim 8, wherein, for each of the top blank and the bottom blank, the distance from the loading zone to the boundary of the overlap zone is at least ten millimeters.

10. The weld coupon of claim 9, wherein, for each of the top blank and the bottom blank, the length of the equipotential zone is greater than the distance from the loading zone to the boundary of the overlap zone.

11. The weld coupon of claim 10, wherein for each of the top blank and the bottom blank, the equipotential zone defines a region for application of external probes for measuring potential drop of an electrical current passing through the weld.

12. The weld coupon of claim 11, wherein, for each of the top blank and the bottom blank, the distance from the loading zone to the boundary of the overlap zone is greater than or equal to the width of the weld coupon and less than or equal to twice the width of the weld coupon.

13. The weld coupon of claim 12, wherein a length of the overlap zone is the sum of twice the distance between the boundary of the overlap zone to the weld and a length of the weld.

14. The weld coupon of claim 13, wherein an overall length of each of the top blank and the bottom blank is equal to a sum of the length of the overlap zone, the length of the loading zone and the distance from the loading zone to the boundary of the overlap zone.

15. The weld coupon of claim 14, wherein:
an overall length of the weld coupon is equal to a sum of the length of the overlap zone, the length of the loading zone of the top blank, the length of the loading zone of the bottom blank, the distance from the loading zone of the top blank to the boundary of the overlap zone and the distance from the loading zone of the bottom blank to the boundary of the overlap zone.

16. A method of using a weld coupon for testing weld integrity, wherein the weld coupon includes a top blank and a bottom blank in overlapping engagement with one another and connected to one another with an electrical weld, each of the top blank and bottom blank adapted to uniformly pass an electrical current applied to the weld coupon across the weld and to uniformly pass a tensile load applied to the weld coupon across the weld, the method comprising:
passing an electrical current through the weld coupon and measuring a potential drop of the electrical current across the weld; and
applying a tensile load to the weld coupon.

17. The method of claim 16, wherein the weld coupon further includes:
an overlap zone defined by the overlapping engagement of the top blank and the bottom blank, the electrical weld positioned within the overlap zone;
each of the top blank and the bottom blank including:
a loading zone positioned at an end distal from the weld and having a length that is greater than or equal to both a minimum length adapted to allow tensile loading to be applied to the weld coupon, and a minimum length adapted to allow electrical current to be applied to the weld coupon, and adapted to:
support application of an electrical current from the loading zone of the top blank, across the weld, to the loading zone of the bottom blank; and
support application of a tensile load from the loading zone of the top blank, across the weld, to the loading zone of the bottom blank; and
a distribution zone extending from the weld to the loading zone and adapted to:
uniformly pass applied electrical current between the loading zone and the weld; and
uniformly pass applied tensile load between the loading zone and the weld;
wherein:
a distance from the loading zone to a boundary of the overlap zone adapted to uniformly distribute applied tensile load between the loading zone and the weld across a width of the weld coupon; and
each of the top blank and the bottom blank includes an equipotential zone extending from the loading zone, wherein, within the equipotential zone electrical current applied between the loading zone and the weld is uniformly distributed across the width of the weld coupon;
the passing an electrical current through the weld coupon and measuring a potential drop of the electrical current across the weld further including:
applying a first electrical probe to the top blank at a position adjacent to the boundary of the overlap zone on a first side of the weld;
applying a second electrical probe to the bottom blank at a position adjacent to the boundary of the overlap zone on a second side of the weld;
passing an electrical current between the loading zone of the top blank and the loading zone of the bottom blank, across the weld; and
measuring a potential drop between the first electrical probe and the second electrical probe.

18. The method of claim 17, wherein the distribution zone of each of the top blank and the bottom blank has a length that is greater than or equal to both:
a sum of the distance from the loading zone to the boundary of the overlap zone and a distance from the boundary of the overlap zone to the weld; and
a sum of a length of the equipotential zone and a distance from the equipotential zone to the weld; and
wherein:
the passing an electrical current through the weld coupon and measuring a potential drop of the electrical current across the weld further includes uniformly passing the electrical current applied to the weld coupon across the distribution zone between the loading zone and the weld; and
the applying a tensile load to the weld coupon further includes uniformly passing tensile load applied to the weld coupon across the distribution zone between the loading zone and the weld.

19. A method of forming a weld coupon for testing weld integrity, comprising:
forming a top blank and a bottom blank, wherein each of the top blank and the bottom blank includes a loading zone and a distribution zone; and
attaching, with an electrical weld, the top blank and the bottom blank in overlapping engagement with one another, the overlapping engagement defining an overlap zone of the weld coupon;
for each of the top blank and the bottom blank:
the loading zone adapted to support application of an electrical current from the loading zone of the top blank, across the weld, to the loading zone of the bottom blank, and support application of a tensile load from the loading zone of the top blank, across the weld, to the loading zone of the bottom blank; and
the distribution zone extending from the weld to the loading zone and adapted to uniformly pass applied electrical current between the loading zone and the weld, and uniformly pass applied tensile load between the loading zone and the weld;
the forming the top blank and the bottom blank further including forming the top blank and the bottom blank wherein:
the loading zone has a length that is greater than or equal to both a minimum length adapted to allow tensile loading to be applied to the weld coupon, and a minimum length adapted to allow electrical current to be applied to the weld coupon;
the loading zone of the top blank has a length that is equal to a length of the loading zone of the bottom blank, and the distribution zone of the top blank has a length that is equal to a length of the distribution zone of the bottom blank;
a distance from the loading zone to a boundary of the overlap zone is adapted to uniformly distribute applied tensile load between the loading zone and the weld across a width of the weld coupon;
each of the top blank and the bottom blank includes an equipotential zone extending from the loading zone, wherein, within the equipotential zone electrical current applied between the loading zone and the weld is uniformly distributed across the width of the weld coupon; and
the distribution zone of each of the top blank and the bottom blank has a length that is greater than or equal to both:
a sum of the distance from the loading zone to the boundary of the overlap zone and a distance from the boundary of the overlap zone to the weld; and
a sum of a length of the equipotential zone and a distance from the equipotential zone to the weld.

20. The method of claim 19, wherein the forming the top blank and the bottom blank further includes forming the top blank and the bottom blank, wherein:
for each of the top blank and the bottom blank, the distance from the loading zone to the boundary of the overlap zone is at least ten millimeters;
for each of the top blank and the bottom blank, the length of the equipotential zone is greater than the distance from the loading zone to the boundary of the overlap zone;
for each of the top blank and the bottom blank, the equipotential zone defines a region for application of external probes for measuring potential drop of an electrical current passing through the weld;
for each of the top blank and the bottom blank, the distance from the loading zone to the boundary of the overlap zone is greater than or equal to the width of the weld coupon and less than or equal to twice the width of the weld coupon;
a length of the overlap zone is the sum of twice the distance between the boundary of the overlap zone to the weld and a length of the weld;
an overall length of each of the top blank and the bottom blank is equal to a sum of the length of the overlap zone, the length of the loading zone and the distance from the loading zone to the boundary of the overlap zone; and
an overall length of the weld coupon is equal to a sum of the length of the overlap zone, the length of the loading zone of the top blank, the length of the loading zone of the bottom blank, the distance from the loading zone of the top blank to the boundary of the overlap zone and the distance from the loading zone of the bottom blank to the boundary of the overlap zone.
